(19) United States
(12) Patent Application Publication
(10) Pub. No.: US 2011/0288437 A1
(43) Pub. Date: Nov. 24, 2011

(54) TISSUE SAMPLING DEVICE

(76) Inventor: Shawn RYAN, Upton, MA (US)

(21) Appl. No.: 13/111,570

(22) Filed: May 19, 2011

Related U.S. Application Data
(60) Provisional application No. 61/346,635, filed on May 20, 2010.

(51) Int. Cl. A61B 10/02

(52) U.S. Cl. 600/567

(57) ABSTRACT

A biopsy device includes a longitudinal element extending from a proximal end to a distal end and including a lumen extending therethrough and a stylet extending through the lumen along a longitudinal axis thereof and including a tissue receiving structure formed in a surface thereof, the tissue receiving structure being located so that, when the device is in a closed configuration, the tissue receiving structure faces an inner wall of the longitudinal element. The stylet is movable between a tissue capture configuration in which the tissue receiving structure is exposed to receive target tissue and the closed configuration in combination with an actuator coupled to one of the stylet and the longitudinal element for moving one of the stylet and the longitudinal element proximally while the other of the stylet and the longitudinal element is held substantially stationary to sever tissue received within the tissue receiving cavity from surrounding tissue.

### PRIORITY CLAIM

This application claims the priority to the U.S. Provisional Application Ser. No. 61/346,635, entitled “Tissue Sampling Device” filed May 20, 2010. The specification of the above-identified application is incorporated herewith by reference.

### BACKGROUND

Needle biopsies are common for the diagnosis and the staging of disease. One type of biopsy procedure is endoscopic ultrasound-guided fine needle aspiration (EUS-FNA) which involves the insertion of a needle under ultrasound guidance so that the physician may accurately gauge the position of the needle relative to target tissue to be sampled. EUS-FNA procedures are performed to ensure that the correct tissue is sampled while minimizing risk to the patient. However, in certain clinical situations, sample may be difficult to handle or acquire. One type of device currently in use includes a notched stylet which is extended beyond a distal end of the needle so that the notch of the stylet is within a target tissue mass. When the notch of the stylet is positioned as desired, a spring, which may include a gas stored in a gas piston, is released to drive the needle distally over the stylet capturing a sample within the notch. In certain situations, it may not be desirable to have the needle driven distally without direct physician control. Furthermore, such devices often fail to capture a sample. For example, when the needle extends along a tortuous path, the spring actuation may cause buckling of the needle dissipating the energy stored in the spring before it reaches the distal end of the needle. Additionally, reduced column strength may result in buckling of the needle preventing it from moving over the stylet as desired when the needle extends along a tortuous path. This buckling may slow the distal movement of the needle to a level at which tissue is pushed out of the notch instead of being sliced and maintained therein.

### SUMMARY OF THE INVENTION

The present invention is directed a device, comprising a longitudinal element extending from a proximal end to a distal end and including a lumen extending therethrough and a stylet extending through the lumen along a longitudinal axis thereof and including a tissue receiving cavity formed in a surface thereof, the tissue receiving cavity being located so that, when the device is in a closed configuration, the tissue receiving cavity faces an inner wall of the longitudinal element, the stylet being movable between a tissue capture configuration in which the tissue receiving element is exposed to receive target tissue and the closed configuration in combination with an actuator coupled to one of the stylet and the longitudinal element for moving the one of the stylet and the longitudinal element proximally to sever tissue received within the tissue receiving cavity from surrounding tissue.

### BRIEF DESCRIPTION OF THE DRAWINGS

FIG. 1 shows a cross-sectional side view of a distal end of a device according to a first exemplary embodiment of the present invention, in a tissue capture configuration;

FIG. 2 shows a cross-sectional side view of the distal end of the device of FIG. 1, in a closed configuration;

FIG. 3 shows a side view of a distal end of a stylet according to an alternate embodiment of the device of FIG. 1;

FIG. 4 shows a side view of a distal end of a stylet according to another alternate embodiment of the device of FIG. 1;

FIG. 5 shows a side view of a distal end of a stylet according to yet another alternate embodiment of the device of FIG. 1;

FIG. 6 shows a side view of an actuating mechanism according to the first exemplary embodiment of the present invention;

FIG. 7 shows a cross-sectional perspective view of the actuating mechanism of FIG. 6;

FIG. 8 shows a perspective view of an alternate embodiment of a housing of the actuating mechanism of FIG. 6;

FIG. 9 shows a cross-sectional side view of a distal end of a device according to a second exemplary embodiment of the present invention, in a tissue capture configuration;

FIG. 10 shows a cross-sectional side view of the distal end of the device of FIG. 9, in a closed configuration;

FIG. 11 shows a cross-sectional side view of a distal end of a device according to an alternate embodiment of the present invention.

FIG. 12 shows a cross-sectional side view of a device according to another alternate embodiment of the present invention.

FIG. 13 shows a cross-sectional side view of a distal end of the device according to another exemplary embodiment of the present invention, in a tissue capture configuration; and

FIG. 14 shows a cross-sectional side view of the distal end of the device of FIG. 13, in a closed configuration.

### DETAILED DESCRIPTION

The present invention may be further understood with reference to the following description and the appended drawings, wherein like elements are referred to with the same reference numerals. The present invention relates to devices for needle biopsies and, in particular, relates to an improved biopsy device including a mechanically actuated stylet for extracting target cells. Exemplary embodiments of the present invention describe a stylet movable between a tissue capture configuration in which the device collects a sample and a closed configuration in which the stylet is drawn proximally within a needle, covering the collected tissue sample such that the device may be removed from a body and the collected tissue sample examined. Although exemplary embodiments of the present invention describe an EUS-FNA device, it will be understood by those of skill in the art that the present invention may relate to any biopsy device configured for delivery to a target site within a body via insertion through an endoscope, spyscope, another needle or a catheter such as, for example, a biliary catheter, a balloon catheter, a stent delivery catheter, etc.

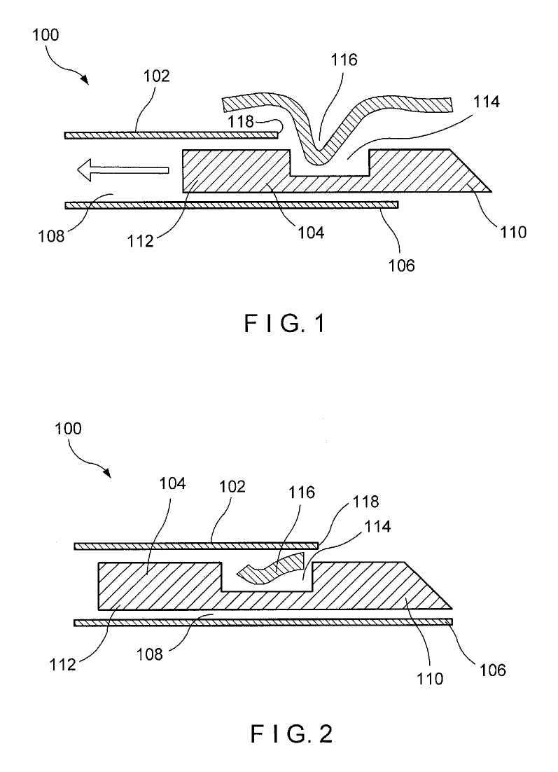

As shown in FIGS. 1-2, a device 100 according to a first exemplary embodiment of the present invention comprises a needle 102 and a stylet 104. The needle 102 extends longitudinally from a tissue penetrating distal tip 106 to a proximal end (not shown) with a lumen 108 extending therethrough. In a first exemplary embodiment, the needle 102 is formed of a hypotube cut at an angle to form the tissue penetrating distal tip 106. The hypotube may be formed of, for example, a polymer, stainless steel, a chromium cobalt alloy, nitinol or any combination of such materials. For example, a distal portion of the needle 102 may be formed of a metal while a proximal portion of the needle 102 is formed of a polymer. It will be understood by those of skill in the art, however, that the needle 102 may be formed of any of a variety of materials so long as the needle 102 is sufficiently flexible to navigate through tortuous paths within the body to the site of the target tissue to be sampled while retaining the column strength necessary to be advanced distally as required to sample tissue. Those skilled in the art will understand that the stylet 104 may be formed as a hollow tube (e.g., a hypotube) with a lumen extending therethrough to the distal end thereof. An opening in a distal portion of the stylet 104 may then be used to conduct negative pressure applied to the lumen of the stylet 104 to, for example, draw target tissue into a desired spatial relation to the stylet 104.

The stylet 104 extends longitudinally within a lumen 108 of the needle 102 from a distal end 110 to a proximal end 112 and is slidable therewithin. The distal end 110 of the stylet 104 in this embodiment is tapered to substantially match a geometry of the distal tip 106 of the needle 102 so that, when the stylet 104 is slid to a closed configuration, the distal opening of the lumen 108 is sealed by the stylet 104 with the distal end 110 forming a substantially smooth surface in relation to the distal tip 106 of the needle 102. The distal end 110, however, may include a variety of tip geometries such as, for example, a sharp tip, a blunt tip or a tapered tip. The stylet 104 includes a notch or groove 114 separated from the distal end 110 so that, when the stylet 104 is in the closed configuration, the groove 114 is housed within the needle 102. The groove 114 extends radially into and longitudinally along a portion of a length of the stylet 104 to form a tissue receiving space within the stylet 104. The groove 114 may also extend around at least a portion of a perimeter of the stylet 104. The stylet 104 is formed of a material which is sufficiently flexible so that, when the stylet 104 is in the closed configuration, the needle 102 may be navigated along a tortuous path to a target tissue site. In a preferred embodiment, the stylet 104 is formed of nitinol and has an outer diameter ranging from between about 0.5 mm and 1.2 mm, and more specifically from between about 0.7 mm and 0.9 mm. It will be understood by those of skill in the art, however, that the stylet 104 may be formed of any size so long as the stylet 104 is insertable through the needle 102 and into a living body.

As shown in FIG. 1, the stylet 104 is movable from the closed configuration to a tissue capture configuration in which the distal end 110 of the stylet 104 extends distally beyond the distal tip 106 of the needle 102 so that the groove 114 is exposed and positioned within a target tissue mass. In the tissue capture configuration, the stylet 104 is preferably extended distally past the distal tip 106 of needle 102 until the entire groove 114 is outside the needle 102 in contact with the target tissue. The tissue naturally compresses around the stylet 104 so that a portion 116 thereof moves into the groove 114. When the user confirms that the portion of tissue 116 is received within the groove 114, the stylet 104 is drawn proximally through the lumen 106 of the needle 102, into the closed configuration so that the portion 116 is cut away from the surrounding tissue by a distal edge 118 of the needle 102 as shown in FIG. 2. As would be understood by those skilled in the art, the distal edge 118 may be sharpened to facilitate cutting of the tissue portion 116. In this position, the portion of tissue 116 is sealed within the tissue receiving space of the groove 114 by the inner wall of the needle 102 so that the portion 116 may be withdrawn from the body for analysis. As the stylet 104 is moved proximally into the needle 102 to capture the tissue portion 116, axially compressive forces are applied to the needle 102 at the distal end and the forces applied to the stylet 104 are tensile. That is, the application of tension to move the stylet 104 while holding the position of the needle 102 constant, minimizes buckling and other problems associated with devices that push the needle distally to capture tissue and the distal edge 118 of the needle more efficiently cuts the portion 116 from the surrounding tissue.

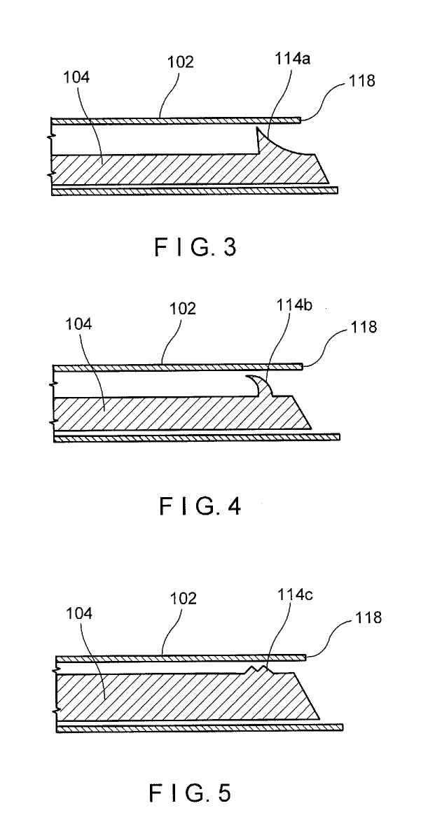

The stylet 104 may include any of a variety of other tissue capturing features along a surface thereof, other than the groove 114. For example, the stylet 104 may include a barb 114a, as shown in FIG. 3, a hook 114b, as shown in FIG. 4, or a roughened surface 114c, as shown in FIG. 5. It will be understood by those of skill in the art that the tissue capturing feature may be any type of feature along the surface of the stylet 104 that grabs tissue after being inserted into target tissue or passed therethrough so that, as the stylet 104 is later moved proximally relative to the needle 102 into the closed configuration, this tissue is dragged proximally along with the stylet into the needle 102. The distal edge 118 of the needle 102, or any other appropriate cutting surface may then sever the grabbed tissue portion 116 from surrounding tissue so that the portion 116 may be housed within needle 102.

Furthermore, a stylet 104 according to any of these embodiments may be drawn proximally through the needle under direct control of a user through a standard connection of the stylet 104 and needle 102 to a manual mechanism. For example, a handle (not shown) of the device may include a first member (not shown) connected to the stylet 104 with a second member coupled to the needle 102 so that movement of the first member proximally relative to the second member draws the stylet 104 proximally into the needle 102. The user may manually extend and retract the stylet 104 with a desired level or force. In addition, the user may manually extract and retract the stylet 104 multiple times if more than one tissue sample is desired.

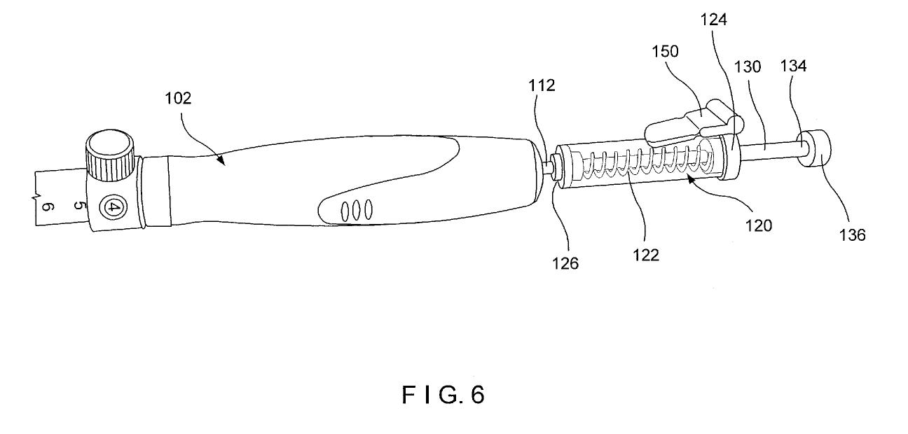

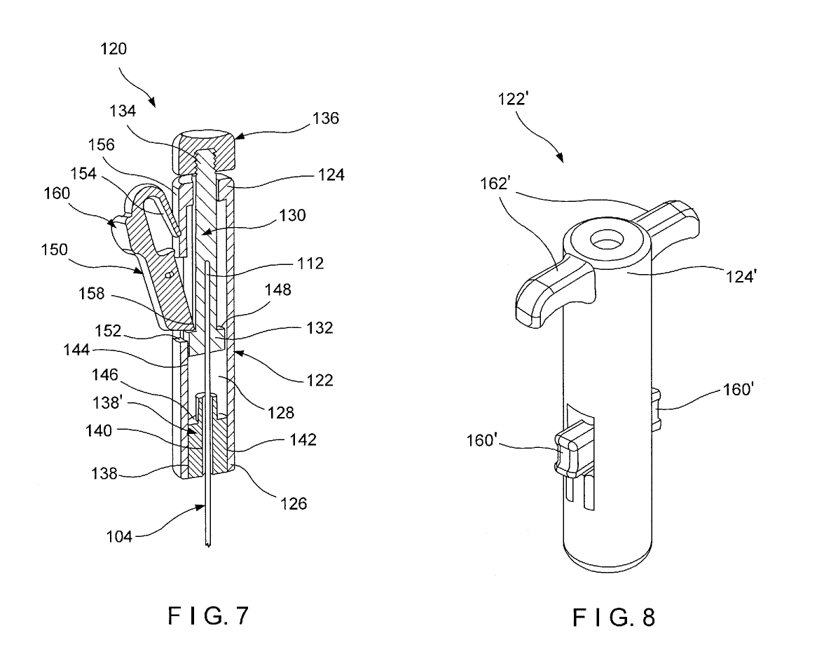

In an alternative embodiment, a mechanism for moving the stylet 104 between the tissue capture configuration and the closed configuration may be an add-on feature or a separate device coupled to a handle of the device 100 described above. For example, as shown in FIGS. 6-7, an actuating mechanism 120 may be attached to the proximal end 112 of the stylet 104 with the stylet 104 inserted through the lumen 108 of the needle 102 as shown in FIG. 6. The actuating mechanism 120, as shown in FIG. 7, comprises a housing 122, a piston 130 and a lever 150. The housing 122 extends from a proximal end 124 to a distal end 126 with a channel 128 extending therethrough. The piston 130 extends from a proximal end 134 to a distal end 132 with the distal end 132 movably housed within the channel 128 while a proximal end 134 extends proximally past the proximal end 124 of the housing 122 to engage a cap 136. The distal end 132 includes a shoulder 148 extending radially outward such that the distal end 132 of the piston is larger than a remaining portion of the piston 130 to engage an inner surface of the channel 128. The distal end 112 of the stylet 104 is fixed to the piston 130 such that moving the piston 130 longitudinally relative to the housing 122 moves the stylet 104 between the tissue capture configuration and the closed configuration.

The actuating mechanism 120 further includes a female luer connection 138 coupling to a male luer 138′ of the needle device. The stylet 104 passes through a lumen 140 of the male luer 138′ into the channel 128. For example, the female luer connection 138 may include a threaded inner surface 144 engaging an outer surface 142 of the male luer 138′. It will be understood by those of skill in the art, however, that the luer 138 may engage the housing 122 in any number of ways. A spring (not shown) may be housed within the channel 128, between a proximal end 146 of the male luer 138′ and the distal end 132 of the piston 130, biasing the piston 130, and thus the stylet 104, toward the closed configuration. The spring may be, for example, a helical, coil spring biasing the piston 130. In an alternative embodiment, the spring may include a gas that may be captured in, for example, a gas piston. It will be understood by those of skill in the art that the spring may be any biasing element that biases the piston 130, and thereby the stylet 104, toward the closed configuration.

The lever 150 is pivotably attached to a side of the housing 122 and extends through an opening 152 in a side of the housing 122. The lever 150 includes a biasing element 154 contacting an outer surface 156 of the housing 122 to bias the lever 150 toward a position angled relative to a longitudinal axis of the housing 122 so that an engaging element 158 of the lever extends through the opening 152 and engages a side of the piston 130. When the actuating mechanism 120 is in the closed configuration, the piston 130 is moved proximally through the channel 128 with the engaging element 158 of the lever 150 engaging the radially protruding distal end 132 of the piston. In this position, the lever 150 is rotated to a position substantially parallel to the longitudinal axis of the housing 122 (i.e., substantially parallel to the needle 102 extending therethrough). To move the device to the tissue capture configuration, a user pushes the cap 136 distally toward the proximal end 124 of the housing 122 moving piston 130 distally through the housing 122 until the engaging element 158 of the lever 150 moves out of contact with the radially protruding end 132 of the piston 130. At this point, the biasing element 154 rotates the lever 150 (counterclockwise as seen in FIG. 7) so that the engaging element 158 moves radially inward to engage the reduced diameter portion of the piston 130 seated proximally of the shoulder 148. The spring (not shown) in the channel 128 urges the piston 130 against the engaging element 158, locking the device in the tissue capture configuration. To withdraw the stylet 104 proximally into the closed configuration, the user simply rotates the lever 150 (clockwise as seen in FIG. 7) to disengage the engaging element 158 from the shoulder 148 so that the spring pushes the piston 130 proximally and the stylet 104 is drawn proximally into the needle 102 (i.e., into the closed configuration).

As described above, in the tissue capture configuration, the distal end 110 of the stylet 104 extends distally past the distal tip 106 of the needle 102 so that the groove 114 is exposed and in a tissue capture configuration. Once target tissue 116 is received within the groove 114, a user may press a button 158 on the lever 150 to release the engaging element 156 from the shoulder 148 and allow the piston 130 to move proximally into the closed configuration. This draws the stylet 104 along with the collected target tissue 116 proximally into the lumen 108 of the needle 102.

As shown in FIG. 8, a housing 122′ of a device according to a further embodiment is substantially similar to the housing 122 of the actuating mechanism 120, but further includes ergonomic features to facilitate manipulation by a user. For example, the housing 122′ includes finger grips 160′ at a proximal end 124′ making the housing 122′ more easily gripped as the cap 136 is pressed into the tissue capture configuration. As opposed to the single button of the mechanism 120, the housing 122′ may include dual buttons 160′ positioned on opposing sides of the housing 122′ facilitating the such that the dual buttons 160′ may be easily pressed as the housing 122′ is gripped by the user. It will be understood by those of skill in the art that the dual buttons 160′ will work in substantially the same manner as described above for the button 160 of the lever 150.

Although the device 100 is described as being used with the actuating mechanism 120, it will be understood by those of skill in the art that the device 100 may be moved between the closed and tissue capture configurations using any actuating mechanism capable of drawing the stylet 104 into the lumen 108 of the needle 102.

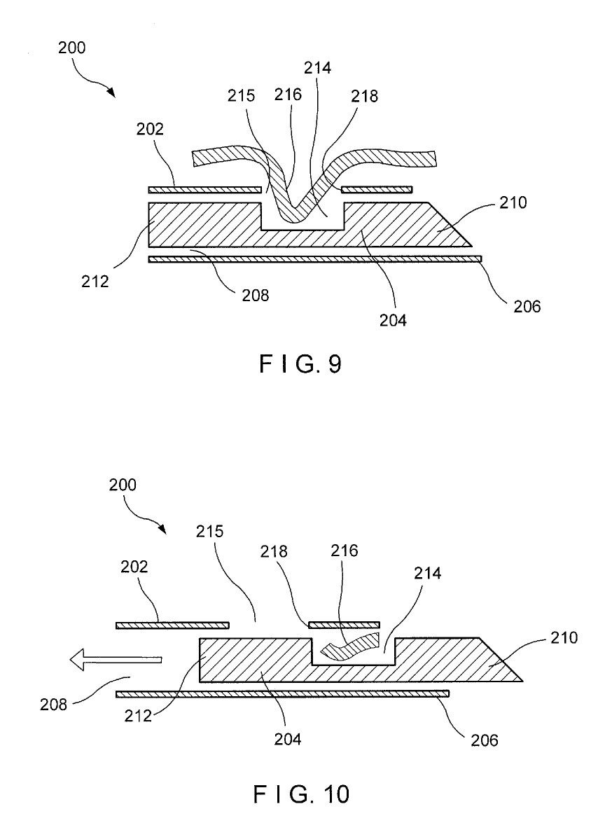

The device 200 according to a further embodiment of the invention as shown in FIGS. 9-10 is substantially similar to the device 100, as described above, except as noted below. The device 200 comprises a needle 202 and a stylet 204. However, rather than moving the stylet 204 between a tissue capture configuration and a closed configuration by drawing the stylet 204 proximally into the needle 202, the needle 202 is drawn in a proximally over the stylet 204 to move the device 200 from the tissue capture configuration to the closed configuration. The stylet 204 is substantially similar to the stylet 104 and extends from a distal end 210 to a proximal end 212 with a groove 214 formed in a radially outer surface thereof. The needle 202 is also substantially similar to the needle 102, extending from a distal end 206 to a proximal end (not shown), but further includes a lateral opening 215 through a surface thereof corresponding to a position of the groove 214 when the device is in the tissue capture configuration.

In the tissue capture configuration, as shown in FIG. 9, the opening 215 extends over the groove 214 opening the groove 214 to the exterior of the device so that tissue 216 may enter the groove 214 via the opening 215. In the closed configuration, as shown in FIG. 10, the needle 202 is moved proximally over the stylet 204 until the groove 214 is covered by a portion of the wall of the needle 202 distal of the opening 215. Thus, tissue 216 received within the groove 214 is covered by the wall of the needle 202 and maintained therewithin for removal from the body. The device 200 may be moved from the tissue capture configuration to the closed configuration via an actuating mechanism similar to the actuating mechanism 120 described above in regard to the device 100. However, rather than being attached to the stylet 204, the actuating mechanism 120 of this embodiment is attached to the needle 202 such that the needle 202 is drawn proximally over the stylet 204 from the tissue capture configuration to the closed configuration while the stylet 204 remains substantially stationary. As the needle 202 is moved proximally from the tissue capture site, a distal edge 218 of the opening 215 cuts the tissue 216 severing it from the surrounding tissue so that the portion of tissue 216 is free to be stored in the groove 214. The distal edge 218 of the opening 215 may be sharpened to facilitate cutting of the tissue portion 216. It will be understood by those of skill in the art that a proximal edge of the opening 215 may also be similarly sharp. The stylet 210 is then drawn proximally relative to the needle 202 so that the sample is captured therewithin for withdrawal from the body.

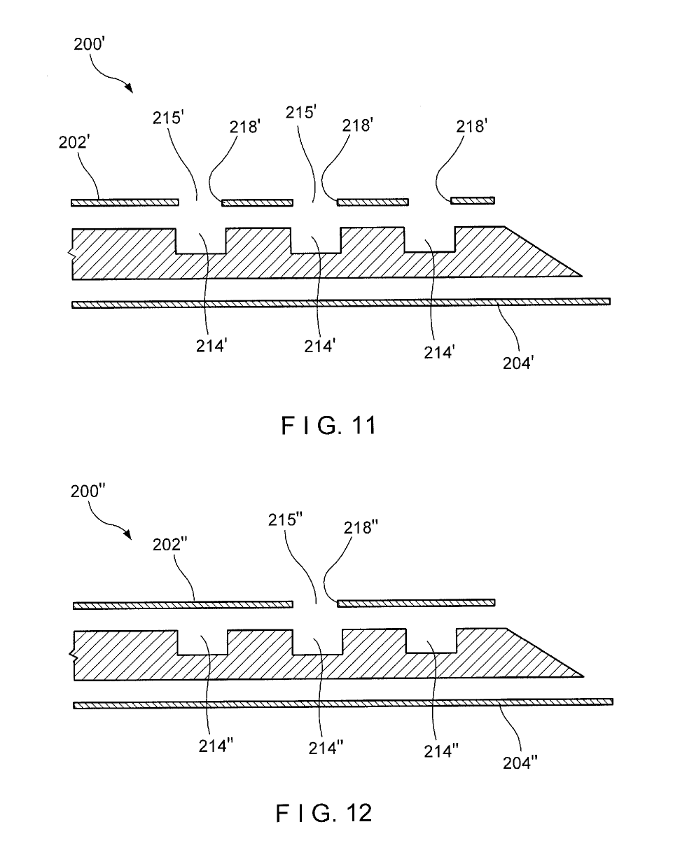

As shown in FIG. 11, a device 200′ according to an alternate embodiment includes a plurality of lateral openings 215′ and corresponding grooves 214′ in a needle 202′ and a stylet 204′, respectively. The device 200′ is substantially similar to the device 200 except that multiple portions of tissue may be sampled simultaneously since each groove 214′ is positioned adjacent to a corresponding opening 215′. Thus, after portions of tissue are received in the openings 215′ and grooves 214′, the needle 202′ is moved proximally to cut the tissue samples at distal edges 218′ of the openings 215′. The stylet 210′ is then withdrawn proximally into the needle 202′ to capture the samples therein for withdrawal from the body. In another embodiment, as shown in FIG. 12, a device 200″ comprises a plurality of grooves 214″ in a stylet 204″, but only one lateral opening 215″ in a needle 202″ so that different portions of tissue may be sampled sequentially. An edge 218″ of the opening 215″ may be used multiple times to collect tissue samples in each of the grooves 214″. It will be understood by those of skill in the art that the tissue samples are collected in each of the multiple grooves 214′ and 214″ of the devices 200′ and 200″, respectively, substantially as described above in regard to the device 200.

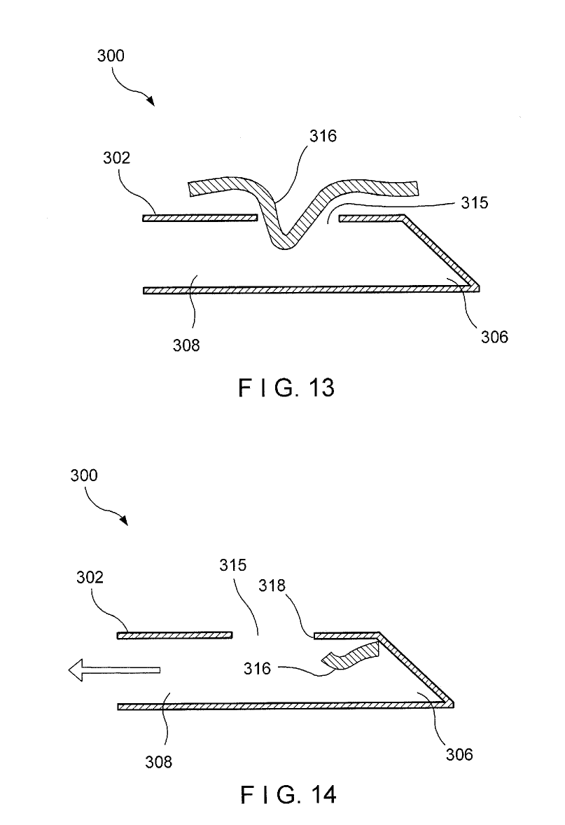

As shown in FIGS. 13-14, a device 300 according to a further embodiment of the invention is substantially similar to the device 200 described above except for the absence of a stylet as noted below. The device 300 includes a needle 302 with a closed distal end 306 and a lateral opening 315 into which target tissue enters. After a target tissue mass has been identified, the needle 302 is extended distally from an endoscope or other insertion device (not shown) into the target tissue mass as shown in FIG. 10 until pressure from the surrounding tissue moves target tissue 316 into the lumen 308 of the needle 302 via the opening 315. If desired, vacuum pressure may be applied to the lumen 308 to aid in drawing the target tissue 316 into the lateral opening 315. The needle 302 is then drawn proximally through the target tissue mass as shown in FIG. 11 so that a distal edge 318 of the opening 315 slices a portion of the target tissue 316 away from the surrounding tissue. Continued proximal movement of the needle 302 drives the tissue 316 distally into the lumen 308 out of alignment with the opening 315 so that the tissue 316 is maintained in the lumen 318 for removal from the body. The lateral opening 315 and distal edge 318 may be configured such that the lateral opening 315 is filled with the target tissue after a withdraw stroke of approximately 1 cm. It will be understood by those of skill in the art, however, that the withdraw stroke may vary so long as the withdrawal stroke is sufficient to slice and collect the target tissue 316 within the lateral opening 315.

The needle 302 may alternatively be mounted in the same manner as the previously described stylets within a concentric outer tube (not shown). The outer tube may then include a distal tissue cutting edge so that, after tissue has been drawn into the lateral opening 315, the needle 302 may be drawn proximally into the outer tube so that the distal tissue cutting edge thereof slices the target tissue from surrounding tissue so that the severed tissue is captured within the needle 302 within the outer tube. Those skilled in the art will understand that the hollow needle 302, which maybe formed, for example, as a length of hypotube, may be connected to a source of vacuum pressure to aid in drawing target tissue into the lateral opening 315.

It will be understood by those of skill in the art that the needle 302 may be moved proximally and distally through the insertion device manually or via an actuating mechanism similar to the actuating mechanism 320, described above in regard to the device 100. However, this actuating mechanism will be attached to the needle 302 as opposed to the stylet of the earlier embodiment.

It will be apparent to those skilled in the art that various modifications and variations can be made in the structure and methodology of the present invention, without departing from the spirit or scope of the invention. Thus, it is intended that the present invention cover the modifications and variations of this invention provided that they come within the scope of the appended claims and their equivalents.

What is claimed is:

1. A biopsy device, comprising:
a longitudinal element sized and shaped for insertion to a target location within a living body, the longitudinal element being sufficiently flexible to be inserted to the target location along a tortuous path, the longitudinal element extending from a proximal end to a distal end and including a lumen extending therethrough;
a stylet extending through the lumen along a longitudinal axis thereof and including a tissue receiving structure formed in a surface thereof, the tissue receiving structure being located so that, when the device is in a closed configuration, the tissue receiving cavity structure an inner wall of the longitudinal element, the stylet being movable between a tissue capture configuration in which the tissue receiving structure is exposed to receive target tissue and the closed configuration; and
an actuator coupled to one of the stylet and the longitudinal element for moving the one of the stylet and the longitudinal element proximally to sever tissue received within the tissue receiving cavity from surrounding tissue while the other of the stylet and the longitudinal element is held substantially stationary.

2. The biopsy device of claim 1, wherein the tissue receiving structure comprises a groove formed in a radially outer surface of the stylet extending substantially parallel to the longitudinal axis.

3. The biopsy device of claim 1, wherein the tissue receiving structure comprises a groove extending around at least a portion of a perimeter of the stylet.

4. The biopsy device of claim 1, wherein the one of the stylet and the longitudinal element is the stylet and wherein the longitudinal element includes a tissue cutting edge at a distal end thereof to sever tissue received by the tissue receiving structure from surrounding tissue.

5. The biopsy device of claim 1, wherein the tissue receiving structure is a tissue receiving cavity and wherein the longitudinal element includes a lateral opening in a wall thereof which, when the stylet is in the tissue capture configuration, aligns with the tissue receiving cavity to expose the tissue receiving cavity to tissue surrounding the longitudinal element.

6. The biopsy device of claim 5, wherein a proximal edge of the lateral opening forms a tissue cutting edge to sever tissue received in the tissue receiving cavity from surrounding tissue.

7. The biopsy device of claim 1, wherein one of the longitudinal element and the stylet includes a tissue piercing distal tip.

8. The biopsy device of claim 1, wherein the tissue receiving structure is separated from a distal end of the stylet by a distance selected so that, when the stylet is in the closed configuration, a distal end of the stylet is received within a distal opening in a distal end of the longitudinal element to seal the distal opening.

9. The biopsy device of claim 1, wherein the actuator includes a piston coupled to the one of the longitudinal element and the stylet for movement therewith, the piston being movably received within a handle of the device and being coupled to a biasing member biasing the piston toward a position corresponding to the closed configuration.

10. The biopsy device of claim 9, wherein the one of the longitudinal element and the stylet is the stylet and wherein the actuator includes a latch mechanism for locking the stylet in the tissue capture configuration until released by a user to move the stylet proximally to the closed configuration by the biasing member.

11. The biopsy device of claim 1, wherein the longitudinal element includes a distal end within which a tissue sample is stored.

12. The biopsy device of claim 1, wherein the stylet includes a lumen extending longitudinally therethrough to an opening adjacent to a distal end thereof.

13. A method for obtaining a tissue sample, comprising:
inserting a flexible biopsy device to a target location within a living body along a tortuous path, the biopsy device including a longitudinal element including a lumen extending therethrough and a stylet slidably received within the lumen, the stylet including a tissue receiving structure formed in a surface thereof;
moving the device to a tissue capture configuration in which the tissue receiving structure is exposed to receive target tissue; and
operating an actuator to move one of the stylet and the longitudinal element proximally to a closed configuration in which the tissue receiving structure faces an inner wall of the longitudinal element severing tissue received therein from surrounding tissue and capturing the severed tissue within the longitudinal element while the other of the stylet and the longitudinal element is held substantially stationary.

14. The method of claim 13, wherein the actuator is on a handle of the device which remains external to the living body.

15. The method of claim 13, wherein a distal-facing edge of the longitudinal element forms a tissue cutting blade for severing the target tissue from surrounding tissue.

16. The method of claim 13, wherein the actuator moves the stylet proximally relative to the tissue received by the tissue receiving structure and wherein the longitudinal element includes a distal facing tissue cutting edge at a distal end thereof to sever tissue received by the tissue receiving structure from surrounding tissue as the stylet is withdrawn proximally thereinto.

17. The method of claim 13, wherein operation of the actuator releases a biasing member coupled to a piston coupled to the stylet so that movement of the piston draws the stylet proximally to the closed configuration.

18. The method of claim 17, further comprising releasing a latch mechanism of the actuator which locks the stylet in the tissue capture configuration to move the stylet proximally to the closed configuration.

19. The method of claim 13, further comprising applying suction to a lumen within the stylet so that the suction draws target tissue toward a distal opening of the lumen formed in a distal portion of the stylet.

20. A biopsy device, comprising:
a flexible longitudinal element for insertion along a tortuous path to a target location within a living body, the longitudinal element extending from a proximal end to a distal end and including a lumen extending therethrough;
a stylet extending through the lumen along a longitudinal axis thereof and including a tissue grabbing element formed on a distal portion thereof, the stylet being movable between a tissue capture configuration in which the tissue grabbing element is exposed to receive target tissue and a closed configuration in which the tissue grabbing element is housed within the longitudinal element; and
an actuator coupled to the stylet for moving the stylet proximally relative to the longitudinal element while the other of the stylet and the longitudinal element is held substantially stationary to sever tissue grabbed by the by the tissue grabbing element from surrounding tissue and capture the severed tissue within the longitudinal element.

21. The biopsy device of claim 18, wherein the tissue grabbing element is a barb.

22. The biopsy device of claim 18, wherein the tissue grabbing element is a hook.

23. The biopsy device of claim 18, wherein the tissue grabbing element is formed as a roughened portion of an outer surface of a distal portion of the stylet.
